(19) United States
(12) Patent Application Publication
(10) Pub. No.: US 2025/0000343 A1
(43) Pub. Date: Jan. 2, 2025

(54) IMAGING INSTRUMENT AND METHODS OF IMAGING A TARGET LOCATION

(71) Applicant: Intuitive Surgical Operations, Inc., Sunnyvale, CA (US)

(72) Inventors: Ralf KLEISER, Freiburg (DE); Florian WIRTH, Lahnau (DE)

(73) Assignee: Intuitive Surgical Operations, Inc., Sunnyvale, CA (US)

(21) Appl. No.: 18/754,795

(22) Filed: Jun. 26, 2024

Related U.S. Application Data
(60) Provisional application No. 63/523,978, filed on Jun. 29, 2023.

(51) Int. Cl. A61B 1/00; A61B 1/05; A61B 1/07

(52) U.S. Cl. CPC A61B 1/00188; A61B 1/00042; A61B 1/00066; A61B 1/00096; A61B 1/051; A61B 1/07

(57) ABSTRACT

Systems and methods are provided for imaging a target location. Accordingly, an imaging instrument includes an imaging system positioned within an elongated body. The imaging system includes an optics chamber that is hermetically sealed within the elongated body. A focusing element is movably positioned within the optics chamber. An image sensor is operably coupled to the focus element. A focus driver is positioned within the optics chamber and operably coupled to the focusing element. The focus driver is configured to move a longitudinal position of the focusing element in response to control input. Additionally, the imaging system includes a seal member that is configured to maintain the hermetic seal of the optics chamber while the control input affects the focus driver.

### CROSS-REFERENCE TO RELATED APPLICATIONS

This patent application claims priority to and the filing date benefit of U.S. Provisional Patent Application No. 63/523,978, filed Jun. 29, 2023, entitled “IMAGING INSTRUMENT AND METHODS OF IMAGING A TARGET LOCATION,” which is incorporated herein by reference in its entirety.

### BACKGROUND

The embodiments described herein relate to remote imaging of a target location, such as in the execution of a minimally invasive medical procedure. More particularly, the embodiments described herein relate to an imaging instrument and methods for imaging the target location with the imaging instrument.

Minimally invasive medical techniques are aimed at reducing the amount of extraneous tissue that is damaged during diagnostic or surgical procedures, thereby reducing patient recovery time, discomfort, and deleterious side effects. For example, cannula sleeves can be passed through incisions in a patient's abdominal wall to provide entry ports through which various instruments can be passed. The incisions are sized to accommodate the cannula sleeves. Insofar as it is desirable to minimize the size of the incisions used to access the target location to reduce the amount of extraneous tissue that is damaged, it is, therefore, desirable minimize the diameter of the cannula sleeve. Minimizing the diameter of the cannula sleeve, in turn, limits the diameter of any instrument to be inserted through the cannula sleeve.

Known minimally invasive techniques, such as Minimally Invasive Surgery (MIS), employ instruments to manipulate tissue that can be either manually controlled or controlled via hand-held or mechanically grounded teleoperated medical systems that operate with at least partial computer-assistance (“telesurgical systems”). Many known MIS instruments include a therapeutic or diagnostic end effector (e.g., forceps, a cutting tool, or a cauterizing tool) mounted on an optional wrist mechanism at the distal end of a shaft. During an MIS procedure, the end effector, wrist mechanism, and the distal end of the shaft are typically inserted into a small incision or a natural orifice of a patient via a cannula to position the end effector at a work site within the patient's body. The optional wrist mechanism can be used to change the end effector's position and orientation with reference to the shaft to perform a desired procedure at the work site.

The most common form of MIS may be endoscopy. The endoscopic surgical instruments generally include an endoscope (for illuminating and viewing the surgical field) and working tools. In endoscopic surgery, the working tools may be similar to those used in conventional (open) surgery, except that the working end or end effector of each tool is separated from its handle by an extension tube. As used herein, the term end effector means the actual working part of the surgical instrument and can include clamps, graspers, scissors, staplers, and needle holders, for example.

To perform endoscopic surgical procedures, the surgeon passes these working tools or instruments through the cannula sleeves to an internal surgical site and manipulates them from outside the abdomen. The surgeon may monitor the procedure within the internal surgical site by means of an endoscope, also referred to herein an endoscopic camera. MIS procedures employing an endoscopic camera are well known (e.g., arthroscopy, retroperitoneoscopy, pelviscopy, nephroscopy, cystoscopy, cisternoscopy, sinoscopy, hysteroscopy, urethroscopy and the like).

After each surgery, surgical tools (including endoscopic cameras) that are to be reused are generally cleaned, disinfected, and then sterilized in preparation for subsequent use. Surgical tools may be cleaned with water, an enzymatic cleanser, and a scrub brush. Common methods of disinfecting surgical tools involves bathing them in a chemical disinfectant so that the surgical tool, which minimizes the risk of infection in a hospital setting. A surgical tool that is to be reused in another surgery is generally subjected to a further sterilization process where all bacterial are killed so that the tool can be used again for surgery without transmitting bacteria from one patient to another. The sterilization process involves either chemical sterilization techniques or a steam sterilization process using an autoclave.

Disinfection and sterilization by immersion in a chemical liquid may not be as environmentally friendly or efficient as other methods. Disposal of the used chemical is costly and may cause harm to the environment. Another drawback is that the chemicals are generally corrosive, which may damage seals or other components of the instrument. Furthermore, chemical disinfection and sterilization may be slower than other methods. Thus, a surgical instrument may have greater lag time between surgeries.

Similarly, disinfection and sterilization using chemical gases such as ethylene oxide also have their drawbacks. Such gases are highly toxic and/or flammable. Extreme care must be used during and after the disinfection and sterilization process to ensure the safety of both the patient and medical staff. Known disinfection and sterilization using gases may be complicated, and thus may result in greater lag time between surgeries.

Many medical facilities have an autoclave and thus steam sterilization of surgical instruments is commonly employed. Commonly known as autoclave sterilization, this method of sterilization rapidly and effectively sterilizes surgical instruments without toxic chemicals and lengthy procedures. Autoclaving standards vary but two common standards require exposing the instrument to steam at 134 degrees C. at 2 atmospheres for 3 minutes (U.S. Standard) or at this temperature and pressure setting for 18 minutes (European Standard). Autoclaving requires less time than other disinfection methods and does not require the use of toxic chemicals.

In view of the aforementioned, the art is continuously seeking new and improved systems and methods for imaging a target location with an imaging instrument.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

The systems and methods described herein facilitate the imaging of a target location having limited access, such as a surgical site. As described herein, the target location is imaged with an imaging instrument. The imaging instrument includes a hermetically sealed optical chamber and can have a reduced diameter for use in a MIS procedure. For example, the imaging instrument can have an outer diameter of less than 6 mm, which can facilitate access to a target location via a body orifice of a patient. The hermetically sealed optical chamber includes a seal member that maintains the hermetic seal even as a control input is delivered from outside the optical chamber to an imaging system contained within the optical chamber to modify the focus of the imaging system. In some embodiments, the seal member can facilitate a change in the volume of the hermetically sealed optical chamber.

In some embodiments, an imaging system that includes an elongated body that defines a channel. The channel extends longitudinally through the elongated body between a proximal end portion of the elongated body and a distal end portion of the elongated body. The distal end portion of the elongated body is coupled to a distal tip. An imaging system is positioned within the elongated body. The imaging system includes an optics chamber, which is hermetically sealed, and is defined at least in part by a housing. A focusing element is movably positioned within the optics chamber. An image sensor is operably coupled to the focusing element. A focus driver is positioned within the optics chamber and operably coupled to the focusing element. The focus driver is configured to move a longitudinal position of the focusing element in response to a control input. A seal member is coupled to the housing. The seal member is configured to maintain the hermetic seal of the optics chamber while the control input affects the focus driver.

In some embodiments, the imaging instrument includes a fiber-optic bundle positioned within the channel. The fiber-optic bundle includes an input end operably coupled to a light source and an output end positioned at the distal tip. At least a portion of the fiber-optic bundle is configured to be in fluid communication with an environment surrounding the imaging instrument.

In some embodiments, a proximal mechanical assembly is coupled to the proximal end portion of the elongated body. The proximal mechanical assembly is configured to receive the control input. The imaging instrument also includes an actuation member positioned within the elongated body and coupled between the proximal mechanical assembly and the focus driver. The actuation member is configured to apply a mechanical force to the focus driver in a longitudinal direction in response to the control input.

In some embodiments, the seal member is configured to move between a collapsed configuration and an expanded configuration when the control input moves the focus driver between a first focus position and a second focus position. In some embodiments, the seal member is a metal bellows.

In some embodiments, the optics chamber has a first volume when the seal member is in the collapsed configuration and a second volume when the seal member is in the expanded configuration. The second volume is greater than the first volume.

In some embodiments, the housing includes a flange portion. The focus driver includes a proximal portion that extends longitudinally through the flange portion. Additionally, the seal member has a distal end that is coupled to the flange portion and a proximal end that is coupled to the proximal portion of the focus driver.

In some embodiments, the seal member is a sealing assembly. The sealing assembly includes at least one O-ring and a wiper seal. The O-ring and the wiper seal surround either a proximal portion of focus driver or an actuation member.

In some embodiments, an energy storage device is positioned between a portion of the housing and a portion of the focus driver. The energy storage device is configured to exert a biasing force on the focus driver.

In some embodiments, the imaging system includes a set of optical elements positioned to establish an optical axis from a target location, through the focusing element, and onto the image sensor.

In some embodiments, the set of optical elements includes a prism that is positioned between the focus driver and the focusing element.

In some embodiments, the imaging system includes a movable lens holder. The movable lens holder includes a middle portion extending between a distal end and a proximal end. The focusing element is coupled to the distal end of the movable lens holder, the proximal end of the movable lens holder is coupled to the focus driver, and the middle portion of the movable lens holder is positioned radially outward of the prism such that the prism is between the movable lens holder and the image sensor.

In some embodiments, the image sensor is displaced from an optical axis defined by the set of optical elements.

In some embodiments, the set of optical elements includes a sapphire window that is positioned at the distal tip, and the sapphire window defines a portion of the optics chamber.

In some embodiments, the set of optical elements includes a lens group at a fixed longitudinal position that is distal of the focusing element and proximal of the distal tip.

In some embodiments, the elongated body has a maximal outer diameter of less than 6 mm.

In one aspect, the present disclosure is directed to a method of imaging a target location. The method includes orienting a distal tip of an imaging instrument toward the target location. The imaging instrument includes a channel extending longitudinally through an elongated body between a proximal end portion of the elongated body and a distal end portion of the elongated body that is coupled to the distal tip. An imaging system is positioned within the elongated body. The imaging system includes an optics chamber defined at least in part by a housing. The optics chamber is hermetically sealed. A focusing element is movably positioned within the optics chamber. An image sensor is operably coupled to the focusing element. A focus driver is positioned within the optics chamber and operably coupled to the focusing element. The focus driver is configured to move a longitudinal position of the focusing element in response to a control input. A seal member is coupled to the housing. The seal member is configured to maintain the hermetic seal of the optics chamber. The method also includes positioning the focusing element at the longitudinal position by providing a control input to the focus driver while maintaining the hermetic seal and directing a portion of light onto the image sensor via the distal tip and the focusing element to image the target location.

In some embodiments, the imaging instrument includes a fiber-optic bundle positioned within the channel. The fiber-optic bundle includes an input end operably coupled to a light source and an output end positioned at the distal tip. The fiber-optic bundle is in fluid communication with an environment surrounding the imaging instrument. Additionally, the method includes delivering illumination to the target location via the fiber-optic bundle.

In some embodiments, the imaging instrument includes a proximal mechanical assembly that is coupled to the proximal end portion of the elongated body and an actuation member that is positioned within the elongated body and coupled between the proximal mechanical assembly and the focus driver. In such embodiments, the method includes providing a control input to the proximal mechanical assembly and maintaining the hermetic seal of the optics chamber while applying a mechanical force to the focus driver in a longitudinal direction via the actuation member in response to the control input.

In some embodiments, the longitudinal position of the focusing element is a first longitudinal position and the imaging system has a first focus distance when the focus driver is at a first focus position, and the mechanical force is a tension force. Additionally, the method further includes applying the tension force to the focus driver to move the focus driver longitudinally from the first focus position to a second focus position that is proximal to the first focus position. The focusing element is in a second longitudinal position and the imaging system has a second focus distance when the focus driver is at the second focus position, the second focus distance is greater than the first focus distance.

In some embodiments, the method includes moving the focus driver between a first focus position and a second focus position via the mechanical force applied by the actuation member. Additionally, the method includes maintaining the hermetic seal of the optics chamber while transitioning the seal member between a collapsed configuration when the focus driver is at the first focus position and an expanded configuration when the focus driver is at the second focus position.

In some embodiments, the optics chamber has a first volume when the seal member is in the collapsed configuration and a second volume when the seal member is in the expanded configuration. The second volume is greater than the first volume.

In some embodiments, the imaging instrument includes an energy storage device positioned between a portion of the housing and a portion of the focus driver. In such embodiments, the method includes exerting a biasing force on the focus driver in a longitudinal direction. The biasing force positions the focusing element at a default longitudinal position.

In some embodiments, the housing includes a flange portion. The focus driver includes a proximal portion that extends longitudinally through the flange portion. Additionally, the seal member has a distal end that is coupled to the flange portion at a fixed longitudinal position and a proximal end that is coupled to the proximal portion of the focus driver. The proximal portion is movable with the focus driver.

In some embodiments, the imaging system includes a set of optical elements positioned to establish an optical axis from a target location, through the focusing element, and onto the image sensor. The set of optical elements includes a prism that is positioned between the focus driver and the focusing element.

In some embodiments, the imaging system includes a movable lens holder that has a middle portion extending between a distal end and a proximal end. The focusing element is coupled to the distal end of the movable lens holder and the proximal end is coupled to the focus driver. The middle portion of the movable lens holder is positioned radially outward of the prism such that the prism is between the movable lens holder and the image sensor. Accordingly, the method includes applying a force to the proximal end of the movable lens holder via the focus driver at a longitudinal position that is proximal relative to the prism. The method also includes moving the middle portion of the movable lens holder longitudinally relative to the prism to move the focusing element between a first longitudinal position and a second longitudinal position. The focusing element is positioned distally relative to the prism.

### BRIEF DESCRIPTION OF THE DRAWINGS

FIG. 1 is a diagrammatic view of a portion of an imaging instrument according to an embodiment.

FIG. 2 is a perspective view of an imaging instrument according to an embodiment.

FIG. 3 is an end view of the distal end portion of the imaging instrument of FIG. 2.

FIG. 4 is a perspective view of a portion of the imaging instrument of FIG. 2 with an elongated body removed.

FIG. 5 is a cross-sectional side view of a distal portion of the imaging instrument of FIG. 2 taken along the line X-X in FIG. 3 depicting an imaging system in a first focus state.

FIG. 6 is a perspective view of an optics chamber of the imaging instrument of FIG. 2.

FIG. 7 is a cross-sectional side view taken along the line X-X in FIG. 3 of the distal portion of the imaging instrument of FIG. 2 in the same configuration as FIG. 5 depicting the optics chamber having a first volume.

FIG. 8 is a cross-sectional side view of a distal portion of the imaging instrument of FIG. 2 taken along the line X-X in FIG. 3 depicting an imaging system in a second focus state.

FIG. 9 is a side view of the imaging system of the imaging instrument of FIG. 2 with a portion of the housing removed.

FIG. 10 is an exploded view of the components of the imaging system of the imaging instrument of FIG. 2.

FIG. 11 is a perspective view of a portion of the components of the imaging system of the imaging instrument of FIG. 2.

FIG. 12 is a diagrammatic view of a proximal portion of an imaging instrument according to an embodiment.

FIG. 13 is a flow chart of a method of imaging a target location with an imaging instrument according to an embodiment.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

Generally, the present disclosure is directed to systems and methods for imaging a target location, which can be in a space constrained environment. The target location can, for example, be a surgical site that is accessed through an opening (e.g., a body orifice, an incision, and/or a cannula) that has an inner diameter of less than 9 mm (e.g., less than 7 mm). In order to image the target location, the imaging instrument must be sized to pass through the opening. For example, the imaging instrument can be sized to have a maximal diameter that is less than 6 mm. Within these size constraints it remains desirable to illuminate the target location and retain the ability to adjust the focus of the imaging system of the imaging instrument without altering the position of the imaging instrument. Further, as the imaging instrument can be exposed to moisture while imaging the target location and/or during post-procedure processing, it is also desirable that the various optical elements of the imaging instrument be hermetically isolated. Specifically, it is desirable that the imaging instrument be configured to maintain the hermetic seal surrounding the optical elements while receiving the control input that adjusts the focus of the imaging system. Accordingly, the various systems and methods described herein facilitate the establishment and maintenance of a hermetic seal surrounding at least the optical elements in an imaging instrument sized to access the target location.

As described herein, the imaging instrument can include an imaging system that is positioned within an elongated body (e.g., an instrument shaft). For example, the imaging system can be positioned within a distal end of the elongated body. The imaging system can include a set of optical elements (e.g., lenses and prisms) positioned within a hermetically sealed optics chamber. In order to maintain the hermetic seal, a seal member can be coupled to the housing of the optics chamber and to maintain the hermetic seal as a control input is received. The optical elements can include a focusing element that is movably positioned within the optics chamber and operably coupled to an image sensor (i.e., a sensor that converts light waves into an electrical signal). In other words, the optical elements can be positioned to direct reflected light from the target location onto the image sensor. A focus driver can be positioned within the optics chamber to move the focusing element to change the focus of the imaging system without necessitating a movement of the imaging instrument. The focus driver can, for example, be coupled to an actuation member that transfers a mechanical force from a user of the optical instrument to move the focusing element. Due to the seal member, this mechanical force can be transferred to the focus driver without disrupting the hermetic seal of the optics chamber. Accordingly, the contents of the hermetically sealed optics chamber, including the movable focusing element, are isolated from moisture that could degrade (e.g., due to fogging, condensation, and/or other similar effects) the image of the target location.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site (i.e., the target location), and the word “proximal” refers to a direction away from the work site. Thus, for example, the end of an instrument that is closest to the target location would be the distal end of the instrument, and the end opposite the distal end (i.e., the end manipulated by the user) would be the proximal end of the instrument.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial device positions and orientations. The combination of a body's position and orientation define the body's pose (e.g., a kinematic pose).

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, instrument, and variants thereof, can be interchangeably used.

The various imaging instruments described herein can be employed as a handheld instrument or can be used via a teleoperated surgical system. A teleoperated surgical system (“system”) is a system that operates with at least partial computer assistance (a “telesurgical system”). Both the telesurgical system and its components are considered medical devices. Telesurgical system can be a Minimally Invasive Robotic Surgical (MIRS) system used for performing a minimally invasive diagnostic or surgical procedure on a patient who is lying on an operating table. The system can have any number of components, such as a user control unit for use by an operator of the system, such as a surgeon or other skilled clinician, during the procedure. The MIRS system can further include a manipulator unit (popularly referred to as a surgical robot) and an optional auxiliary equipment unit. The manipulator unit can include an arm assembly and a surgical instrument tool assembly removably coupled to the arm assembly. The manipulator unit can manipulate at least one removably coupled medical instrument (instrument) through a minimally invasive incision in the body or natural orifice of the patient while the surgeon views the surgical site and controls movement of the instrument through a control unit. An image of the surgical site is obtained by the imaging instrument (i.e., an endoscope), which can be manipulated by the manipulator unit to orient the endoscope. The auxiliary equipment unit can be used to process the images of the surgical site for subsequent display to the surgeon through the user control unit.

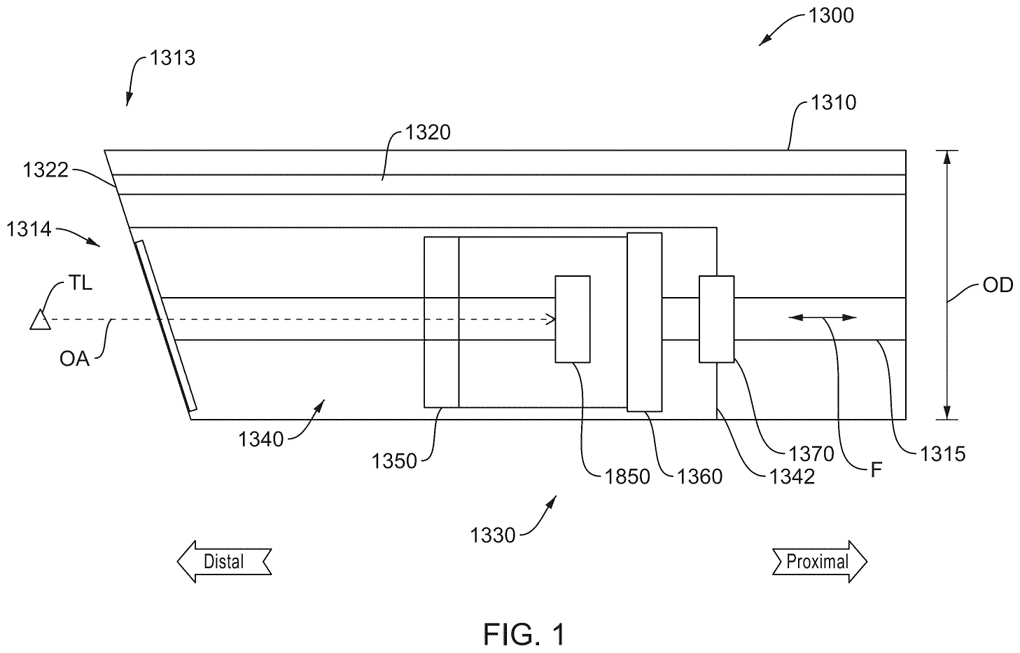

FIG. 1 is a diagrammatic view of a portion of an imaging instrument 1300 according to an embodiment. In some embodiments, the imaging instrument 1300 or any of the components therein can be configured as a handheld endoscope for use in imaging a target location TL (e.g., a surgical site) in a space constrained environment. In some embodiments, the imaging instrument 1300 or any of the components therein are optionally parts of a surgical system that performs minimally invasive surgical procedures and which can include a manipulator unit, a series of kinematic linkages, a series of cannulas, or the like.

In some embodiments, the imaging instrument 1300 can include an elongated body 1310 (e.g., an instrument shaft). The elongated body 1310 defines a channel extending longitudinally through the elongated body 1310 between a proximal end portion of the elongated body 1310 and a distal end portion 1313 of the elongated body 1310. The distal end portion 1313 is coupled to, or forms a portion of, a distal tip 1314. In some embodiments, the elongated body 1310 has a maximal outer diameter OD that is sized to pass through a body orifice and/or a cannula. In other words, the maximal outer diameter OD can be limited by the minimal inner diameter of the body orifice and/or cannula. For example, in order to pass through the body orifice and/or the cannula to image the target location TL, the maximal outer diameter of the elongated body 1310 can be less than 6 mm (e.g., 5.5 mm, 5.0 mm, between 4.5 mm and 6.0 mm, between 5.0 and 6.0 mm, and any suitable range therebetween). It should be appreciated that the limitations on the maximal outer diameter imposed by the necessity to pass through the body orifice and/or the cannula to access the target location TL limits the available volume within the channel available for component placement.

In some embodiments, the imaging instrument 1300 can include an illumination source (e.g., an LED arrangement, a fiber-optic output, a laser illuminator, and/or other similar illuminator) positioned to illuminate the target location TL. For example, a fiber-optic bundle 1320 can be positioned within the channel of the elongated body 1310. The fiber-optic bundle 1320 can include an input end that is operably coupled to a light source and an output end 1322 that is positioned at the distal tip 1314. At least a portion of the fiber-optic bundle 1320 can be configured to be in fluid communication with an environment surrounding the imaging instrument (e.g., the environment at the target location TL). In other words, due in part to the limited maximal outer diameter of the elongated body 1310, in some embodiments, the output end 1322 is not positioned behind a sealed sapphire window. Eliminating the requirement to include a sealed sapphire window to fluidically isolate the illumination source from the target location can facilitate providing an illumination source while complying with the size constraints imposed by the maximal outer diameter OD. In other words, eliminating the sealed sapphire window can allow the diameter of the imaging instrument 1300 to be reduced.

As depicted in FIG. 1, the imaging instrument 1300 includes an imaging system 1330 that is positioned within the elongated body 1310. The imaging system 1330 can, for example, be positioned within the distal end portion 1313 of the elongated body 1310. However, in some embodiments, the imaging system 1330 can extend along the longitudinal length of the elongated body 1310 to the proximal portion.

The imaging system 1330 includes an optics chamber 1340. The optics chamber 1340 is defined, at least in part, by a housing 1342. In order to isolate the components of the imaging system 1330 from moisture at the target location and/or encountered during post-procedure processing, the optics chamber 1340 is hermetically sealed. In other words, the volume defined by the optics chamber 1340 is fluidically isolated from the surrounding environment such that liquids, vapors, and/or gases are precluded from passing between the volume defined by the optics chamber 1340 and the surrounding environment. By maintaining the hermetic seal of the optics chamber 1340, the effect of moisture on the optical components positioned therein is eliminated.

As depicted, the imaging system 1330 includes a focusing element 1350 that is movably positioned within the optics chamber 1340. Additionally, an image sensor 1850, which can be a sensor (e.g., a charge-coupled device or an active-pixel sensor) that converts light waves into an electrical signal, is operably coupled to the focusing element 1350. Accordingly, an optical axis OA extends from the target location to the distal tip 1314, through the focusing element 1350, and on to the image sensor 1850. The focusing element 1350 can facilitate alterations to the focal distance of the imaging system 1330. In order to adjust the focus of the imaging system 1330 by moving the focusing element 1350, in some embodiments, a focus driver 1360 is positioned within the optics chamber 1340. The focus driver 1360 is operably coupled to the focusing element 1350 and configured to move a position (e.g., a longitudinal position or a rotational position) of the focusing element 1350 in response to a control input while the image sensor 1850 remains in a fixed longitudinal position relative to the elongated body 1310. In some embodiments, a portion of the focus driver 1360 is positioned proximal to the image sensor 1850 and configured to receive the control input from an actuation member 1315. The actuation member 1315 is positioned within the elongated body 1310 and can be configured to apply a mechanical force F to the focus driver 1360.

To maintain the hermetic seal, the imaging system 1330 includes a seal member 1370 that is coupled to the housing 1342. The seal member 1370 can, for example, be coupled to a proximal end portion of the housing 1342. The seal member 1370 is configured to maintain the hermetic seal of the optics chamber 1340 while the control input affects the focus driver 1360. In some embodiments, the seal member 1370 surrounds the actuation member 1315, as depicted, or a proximal portion of the focus driver 1360. By surrounding either the actuation member 1315 or the focus driver 1360, the actuation member 1315 can move to exert the mechanical force F on the focus driver 1360 while the seal member 1370 maintains the hermetic seal of the optics chamber 1340. In some embodiments, the seal member 1370 can be configured as a sealing assembly including at least one O-ring (e.g., a high-temperature O-ring such as a polymerized fluoroelastomer dipolymer) and optionally a wiper seal. In some embodiments, the seal member 1370 can be configured as a metal bellows that is configured to move between a collapsed configuration and an expanded configuration in response to the control input.

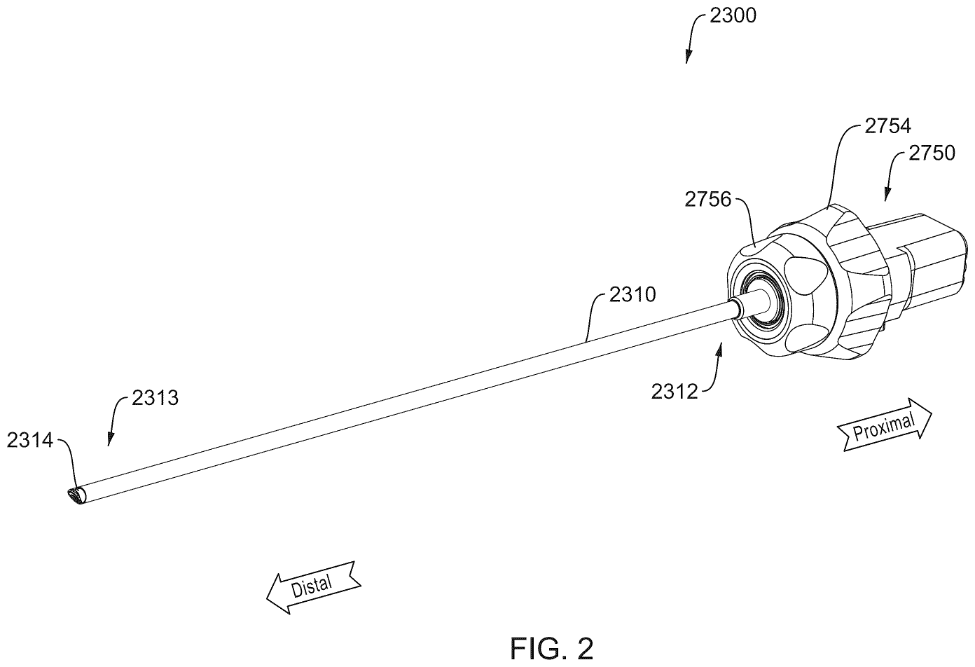

Referring now to FIGS. 2-11, wherein various views of an imaging instrument 2300 according to an embodiment are presented. In some embodiments, the imaging instrument 2300 or any of the components therein can be configured as a handheld endoscope for use in imaging a target location TL (e.g., a surgical site) in a space constrained environment. In some embodiments, the imaging instrument 2300 or any of the components therein are optionally parts of a surgical system that performs minimally invasive surgical procedures and which can include a manipulator unit, a series of kinematic linkages, a series of cannulas, or the like.

FIG. 2 is a perspective view of the imaging instrument 2300. As depicted, the imaging instrument 2300 includes an elongated body 2310 extending longitudinally between a proximal end portion 2312 and a distal end portion 2313. The distal end portion 2313 is coupled to (or forms a portion of) a distal tip 2314. A proximal mechanical assembly 2750 is coupled to the proximal end portion 2312 of the elongated body 2310. The proximal mechanical assembly 2750 is configured to be coupled to a receiving unit (not shown). The proximal mechanical assembly 2750 is also configured to receive a control input from a user of the imaging instrument. For example, in some embodiments, the user of the imaging instrument 2300 can manipulate a first input wheel 2754 to affect an orientation of the distal tip 2314 and/or a second input wheel 2756 to affect a focal distance of the imaging instrument 2300. In some embodiments, the imaging instrument 2300 is affected by the surgical system to image the target location.

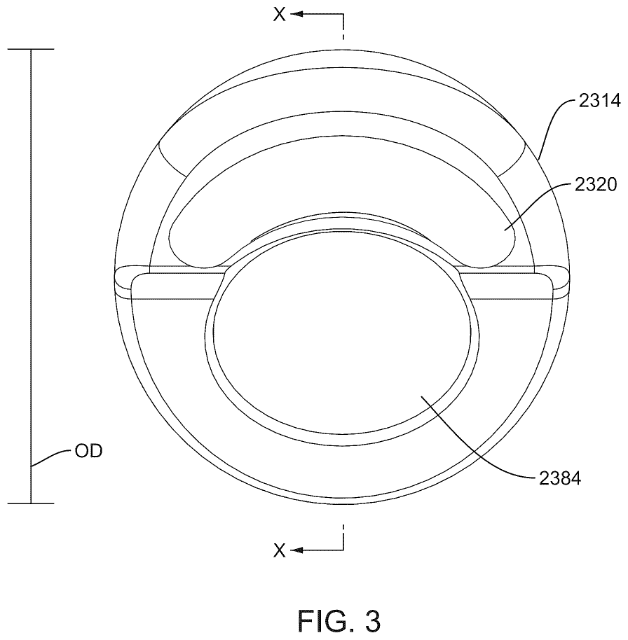

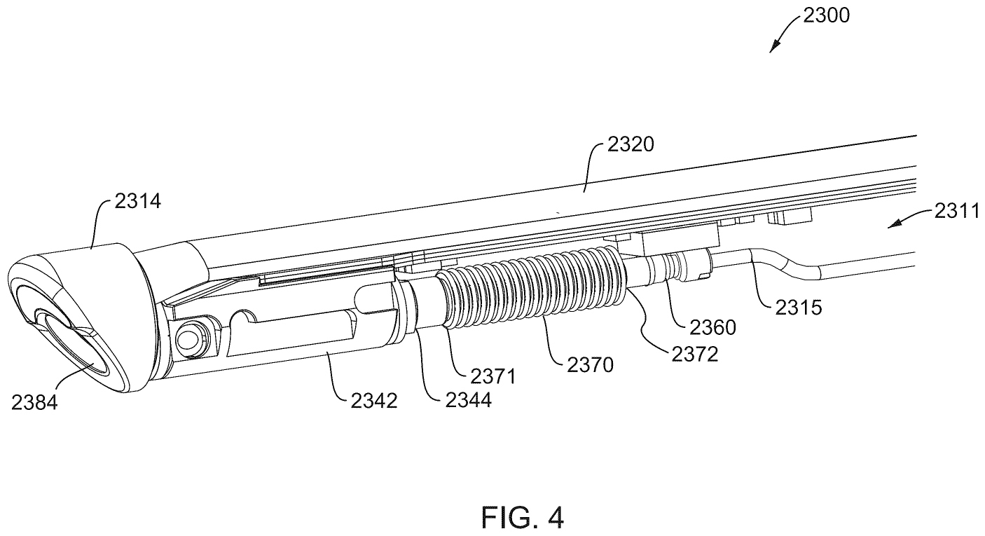

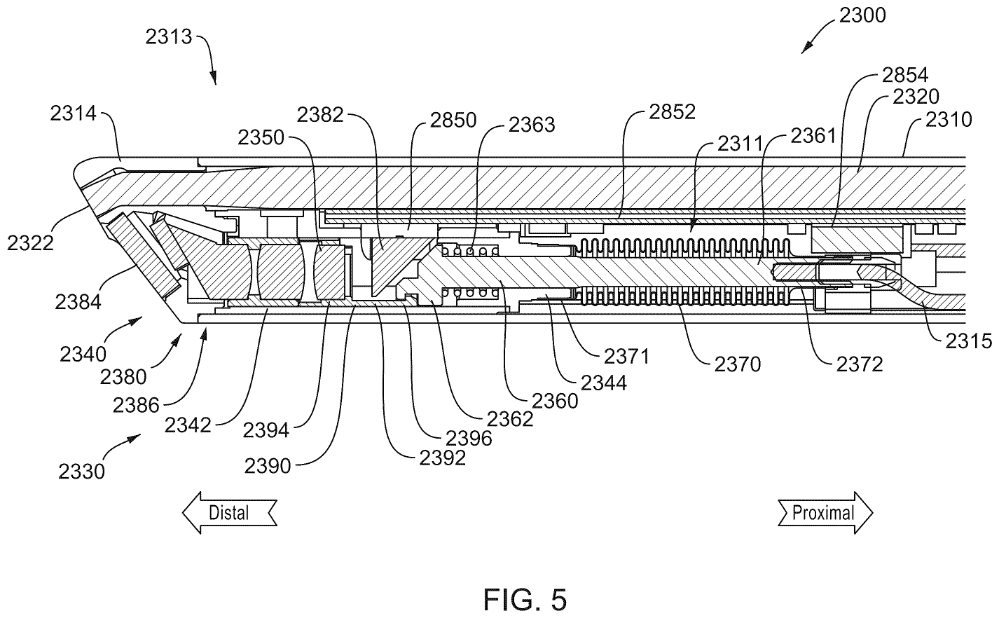

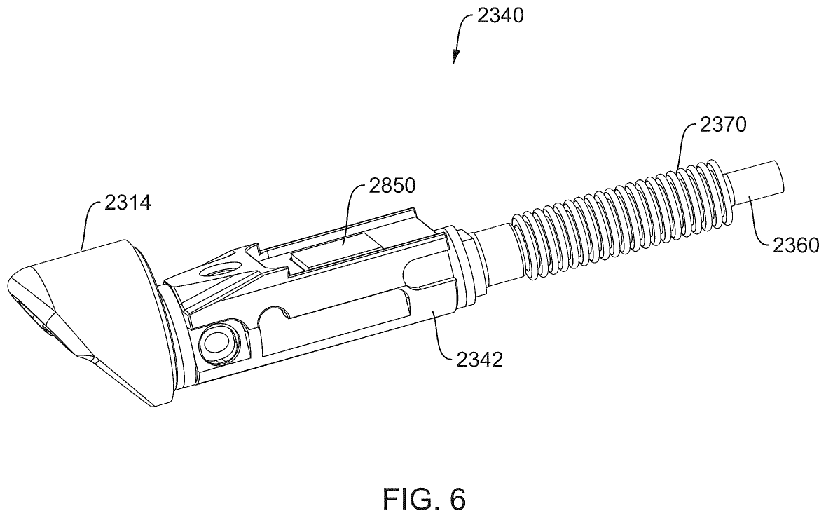

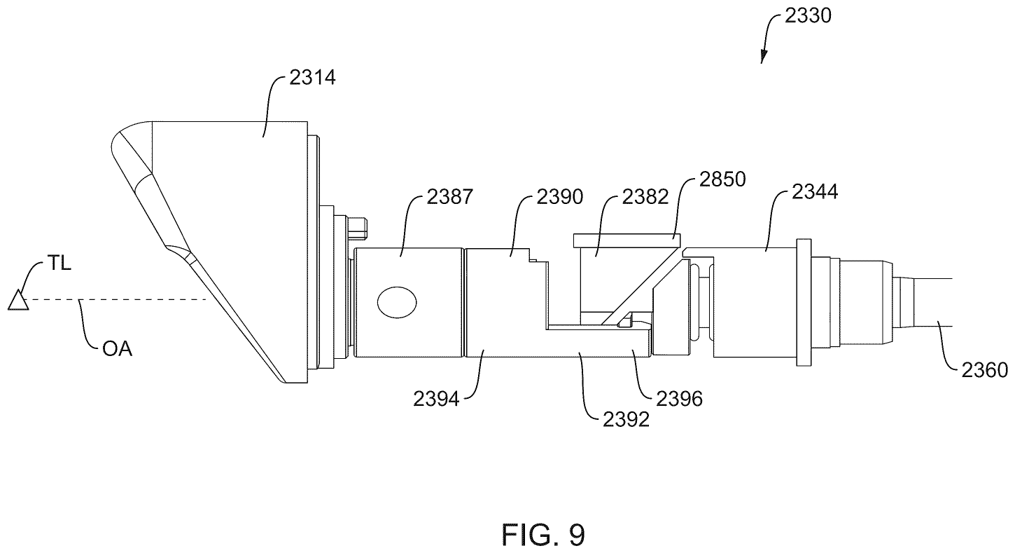

FIG. 3 is an end view of the distal tip 2314 of the imaging instrument 2300, FIG. 4 is a perspective view of the distal end portion 2313 of the imaging instrument 2300 with the elongated body removed, FIG. 5 is a cross-sectional side view of the distal end portion 2313, and FIG. 6 is a perspective view of an optics chamber 2340 of the imaging instrument 2300. As depicted in FIG. 5, the elongated body 2310 defines a channel 2311 that extends longitudinally through the elongated body 2310 between the proximal end portion 2312 and the distal end portion 2313 of the elongated body 2310. The distal end portion 2313 is coupled to a distal tip 2314. In some embodiments, the elongated body 2310 has a maximal outer diameter OD that is sized to pass through a body orifice and/or a cannula. In other words, the maximal outer diameter OD can be limited by the minimal inner diameter of the body orifice and/or cannula. For example, in order to pass through the body orifice and/or the cannula to image the target location TL (FIG. 9), the maximal outer diameter of the elongated body 2310 can be less than 6 mm (e.g., 5.5 mm, 5.0 mm, between 4.5 mm and 6.0 mm, between 5.0 and 6.0 mm, and any suitable range therebetween). It should be appreciated that the limitations on the maximal outer diameter imposed by the necessity to pass through the body orifice and/or the cannula to access the target location TL limits the available volume within the channel 2311 available for an imaging system 2330.

In some embodiments, the imaging instrument 2300 includes a fiber-optic bundle 2320 positioned within the channel 2311 of the elongated body 2310. The fiber-optic bundle 2320 includes an input end that is operably coupled to a light source (not shown), which can be an element of the proximal mechanical assembly 2750. The fiber-optic bundle 2320 also includes an output end 2322 that is positioned at the distal tip 2314. At least a portion of the fiber-optic bundle 2320 can be configured to be in fluid communication with the target location TL. In other words, due in part to the limited maximal outer diameter of the elongated body 2310, the output end 2322 is not positioned behind a sealed sapphire window or other fluidic barrier. Eliminating the requirement to include a sealed sapphire window or other similar structure to fluidically isolate the output end 2322 from the target location TL facilitates providing illumination while complying with the size constraints imposed by the maximal outer diameter OD of elongated body 2310.

In some embodiments, the imaging instrument 2300 includes an imaging system 2330 that is positioned within the elongated body 2310. The imaging system 2330 can, for example, be positioned within the distal end portion 2313 of the elongated body 2310. However, in some embodiments, the imaging system 2330 can extend along the longitudinal length of the elongated body 2310 to the proximal portion.

The imaging system 2330 includes an optics chamber 2340. The optics chamber 2340 (i.e., the boundary of the optics chamber 2340) is defined, at least in part, by a housing 2342. In some embodiments, the optics chamber 2340 can, as more fully described below also be defined by the distal tip 2314, the sapphire window 2384, an image sensor 2850, a seal member 2370, the focus driver 2360 or an actuation member 2315, and the sealed couplings therebetween. The sealed couplings between the various components defining the optics chamber 2340 can, for example, be glue joints, welds, and/or solder joints. In order to isolate the components of the imaging system 2330 positioned therein from moisture at the target location TL and/or encountered during post-procedure processing, the optics chamber 2340 is hermetically sealed. In other words, the volume defined by the optics chamber 2340 is fluidically isolated from the surrounding environment such that liquids, vapors, and/or gases are precluded from passing between the volume defined by the optics chamber 2340 and the surrounding environment. By maintaining the hermetic seal of the optics chamber 2340, the effect of moisture (e.g., fogging or condensation) on the optical components positioned therein is eliminated.

As depicted in FIG. 5, the imaging system 2330 includes a focusing element 2350 that is movably positioned within the optics chamber 2340. Additionally, an image sensor 2850, which can be a sensor (e.g., a charge-coupled device or an active-pixel sensor) that converts light waves into an electrical signal, is operably coupled to the focusing element 2350. Accordingly, an optical path extends from the target location TL to the distal tip 2314, through the focusing element 2350, and on to the image sensor 2850. The focusing element 2350 can facilitate alterations to the focal distance of the imaging system 2330. In order to adjust the focus of the imaging system 2330 by moving the focusing element 2350, in some embodiments, a focus driver 2360 is positioned within the optics chamber 2340. The focus driver 2360 is operably coupled to the focusing element 2350 and configured to move a longitudinal position of the focusing element 2350 in response to a control input while the image sensor 2850 remains in a fixed longitudinal position relative to the elongated body 2310. In some embodiments, a portion of the focus driver 2360 is positioned proximal to the image sensor 2850 and configured to receive the control input from an actuation member 2315. The actuation member 2315 is positioned within the elongated body 2310 and can be configured to apply a mechanical force to the focus driver 2360.

In some embodiments, the actuation member 2315 can be an actuation rod positioned within the elongated body 2310 and coupled between the proximal mechanical assembly 2750 and the focus driver 2360. The actuation member 2315 can be rigid or semi rigid and include a set of bends to conform the actuation member to the channel 2311 of the elongated body 2310. In some embodiments, the actuation member 2315 can be a band or a cable. Being coupled to the proximal mechanical assembly 2750, a control input received by the proximal mechanical assembly 2750 can be transmitted to the imaging system 2330 via the actuation member 2315. For example, in response to the control input presented to the proximal mechanical assembly 2750, the actuation member 2315 can apply a mechanical force to the focus driver 2360. In some embodiments, the mechanical force is applied to the focus driver 2360 in a longitudinal direction.

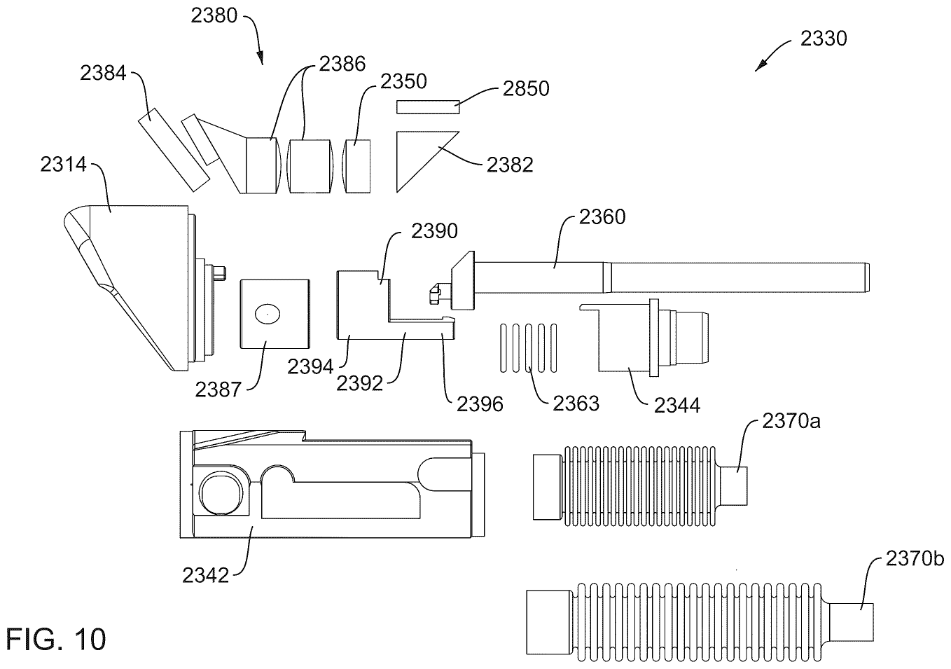

FIG. 9 is a side view of the imaging system 2330 with a portion of the housing 2342 removed for clarity, while FIG. 10 is an exploded view of the imaging system 2330 according to an embodiment. As depicted, the imaging system 2330 includes a set of optical elements 2380. The optical elements 2380 are components of the imaging system 2330 that act on light passing from the target location TL to the image sensor 2850. For example, in some embodiments, the optical elements 2380 include the sapphire window 2384 that is positioned at the distal tip 2314 and hermetically coupled thereto to define a portion of the optics chamber 2340. Additionally, in some embodiments, the optical elements 2380 include a lens group 2386. The lens group 2386 is supported by a lens mounting 2387 and is maintained at a fixed longitudinal position. As depicted the fixed longitudinal position of the lens group 2386 is distal to the focusing element 2350. The fixed longitudinal position of at least a portion of the lens group 2386 is proximal to the distal tip 2314.

The set of optical elements 2380 (e.g., the lens group 2386) are positioned to establish the optical axis OA from the target location TL through the focusing element 2350. Further, the set of optical elements 2380 establishes an optical path from the target location, through the focusing element 2350, and on to the image sensor 2850. The optical path describes the path of travel of a portion of light that is directed onto the image sensor 2850 via the distal tip 2314 and the focusing element 2350. In some embodiments, the image sensor 2850 is displaced from the optical axis OA defined by the optical elements 2380. For example, in some embodiments, the image sensor 2850 can be positioned at a location that is radially outward relative to the optical axis OA. Displacing the image sensor 2850 from the optical axis OA can facilitate the affecting of the longitudinal position of the focusing element 2350 in response to a control input provided to the proximal mechanical assembly 2750.

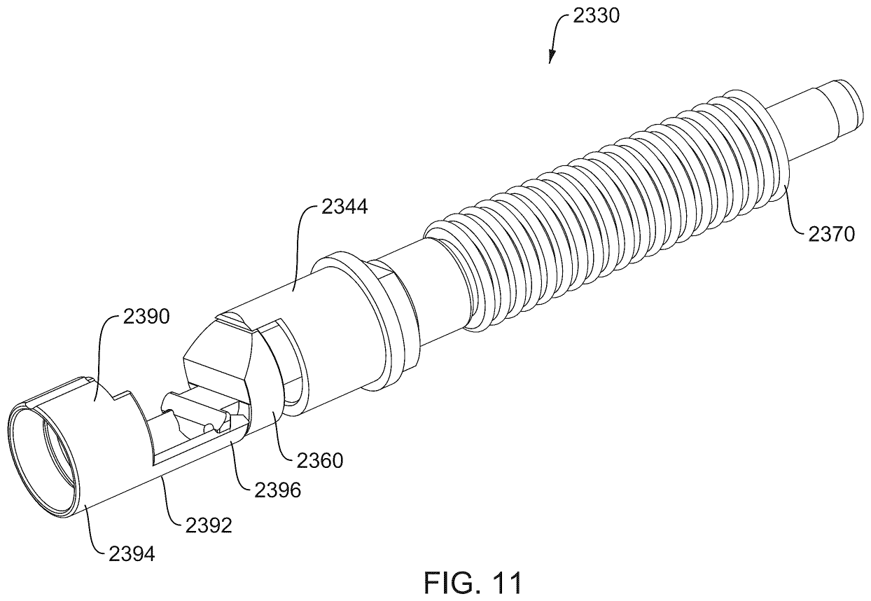

In order to establish the optical path between the target location TL and the image sensor 2850 that is displaced from the optical axis OA, in some embodiments, the set of optical elements 2380 can include a prism 2382. The prism 2382 can be positioned longitudinally between the focus driver 2360 and the focusing element 2350. The prism 2382 can be positioned to divert the portion of light from the optical axis OA and onto the image sensor 2850. As shown, a command input (e.g., the mechanical force), which is received by the focus driver 2360 at a location proximal to the prism 2382, can bypass the prism 2382, which is maintained at a fixed longitudinal position, to affect the longitudinal position of the focusing element 2350. In other words, in response to the control input provided to the proximal mechanical assembly 2750, the actuation member 2315 exerts a force (e.g., a tension force) in the longitudinal direction on the focus driver 2360. The focus driver 2360 transmits the force to the focusing element 2350 via the movable lens holder 2390. The movable lens holder 2390 is positioned within the optics chamber 2340 (e.g., within the housing 2342). In some embodiments, the movable lens holder includes a middle portion 2392 that extends between a distal end 2394 and a proximal end 2396. The focusing element 2350 can, for example, be coupled to the distal end 2394 of the movable lens holder 2390. The proximal end 2396 of the movable lens holder 2390 is coupled to the focus driver 2360, as depicted in FIG. 11. The middle portion 2392 of the movable lens holder 2390 is positioned radially outward of the prism 2382. Said another way, the prism 2382 is between the movable lens holder 2390 and the image sensor 2850. Said yet another way, the middle portion 2392 of the movable lens holder 2390 is positioned between the prism 2382 and the housing 2342 at the longitudinal position of the prism 2382.

In use, the focus driver 2360 transmits the force to the focusing element 2350 via the proximal end 2396 of the movable lens holder 2390 via the coupling therebetween. Insofar as the distal end 2394 of the focus driver 2360 is coupled to the proximal end 2396 via the middle portion 2392, the focusing element 2350 is moved longitudinally by the mechanical force without affecting the longitudinal position of the prism 2382 and the image sensor 2850 optically coupled thereto. It should be appreciated that in alternative the prism 2382 can also be supported by (e.g., coupled to) the movable lens holder 2390 such that the prism 2382 can move along with the focusing element 2350.

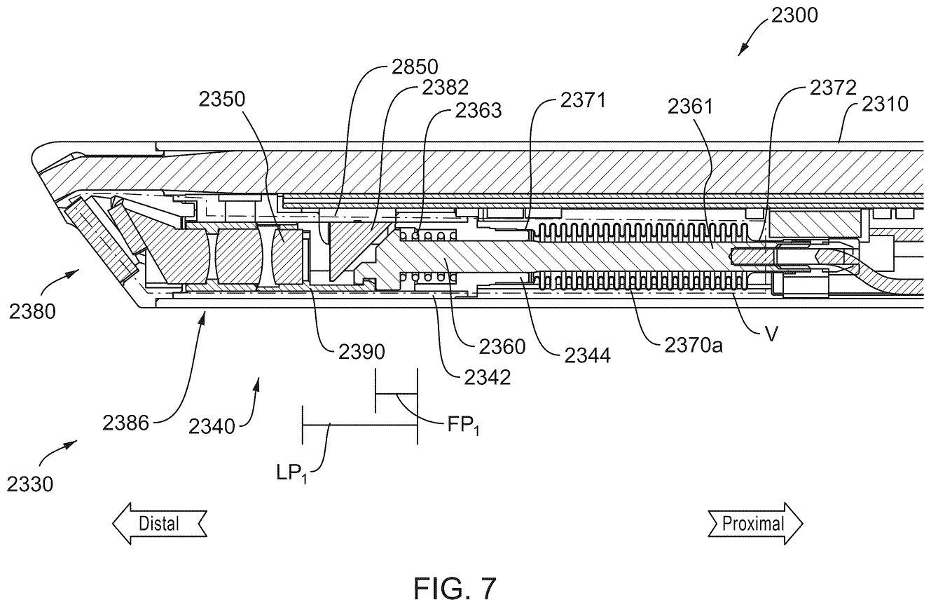

FIG. 7 is a cross-sectional side view of a distal portion of the imaging instrument 2300 in the same configuration as depicted in FIG. 5. As identified in FIG. 7, the focusing element 2350 is positioned at a first longitudinal position LP₁ (with respect to a fixed point on the housing 2342, such as a flange portion 2344) and the focus driver 2360 at a first focus position FP₁ (with respect to a fixed point on the housing 2342, such as a flange portion 2344). On a condition in which the focusing element 2350 is positioned at the first longitudinal position LP₁, the imaging system 2330 has a first focus distance. In some embodiments, the first focus distance is a default focus distance. As such, in some embodiments, first focus position FP₁ maximizes the longitudinal distance between the image sensor 2850 and/or the prism 2382. To establish and/or maintain the default focus distance, in some embodiments, an energy storage device 2363 (e.g., a spring, an elastomeric member, an inflatable member, or other similar structure) is positioned between a portion of the housing 2342 (e.g., a flange portion 2344) and a portion of the focus driver 2360. The energy storage device 2363 can be configured to exert a biasing force on the focus driver 2360. For example, as depicted, the energy storage device 2363 can bias the focus driver 2360, and thus, the focusing element 2350, distally toward the first focus position FP₁. However, in some embodiments, the energy storage device 2363 can be configured to bias the focus driver 2360 proximally.

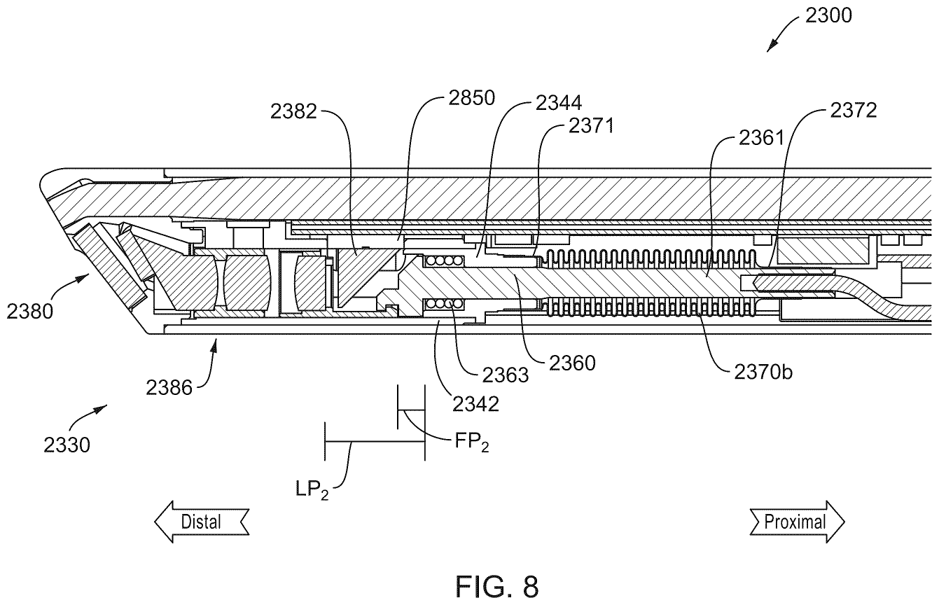

FIG. 8 is a cross-sectional side view of a distal portion of the imaging instrument 2300 with the focusing element 2350 positioned at a second longitudinal position LP₂ and the focus driver 2360 at a second focus position FP₂. On a condition in which the focusing element 2350 is positioned at the second longitudinal position LP₂, the imaging system 2330 has a second focus distance. The second focus position FP₂ is proximal to the first focus position FP₁. The second focus distance is greater than the first focus distance. In other words, the second focus position FP₂ can minimize the longitudinal distance between the image sensor 2850 and/or the prism 2382 thereby increasing the focus distance of the imaging system 2330 and establishing a far-focus distance relative to the default focus distance. Accordingly, the mechanical force exerted on the focus driver 2360 by the actuation member 2315 can be a tension force applied in a proximal direction. The tension force can have a magnitude that is greater than the biasing force of the energy storage device 2363. Thus, the focus driver 2360 can move from the first focus position FP₁ to the second focus position FP₂ in response to the tension force applied by the actuation member 2315 in response to the control input provided to the proximal mechanical assembly 2750. It should be appreciated that in some embodiments, the default position of the focus driver 2360 may correspond to the second focus position FP₂ at which the focus distance for the imaging system 2330 is the far-focus distance and the energy storage device 2363 exerts a tension force on the focus driver 2360. In such embodiments, the actuation member 2315 can be configured to exert a compressive force on the focus driver 2360 to cause the focus driver 2360 to move distally to the first focus position FP₁ to establish a close-focus distance.

To maintain the hermetic seal, the imaging system 2330 includes a seal member 2370 that is coupled to the housing 2342. The seal member 2370 can, for example, be coupled to a proximal end portion of the housing 2342. The seal member 2370 is configured to maintain the hermetic seal of the optics chamber 2340 while the control input affects the focus driver 2360. In some embodiments, the seal member 2370 surrounds the actuation member 2315, or a proximal portion of the focus driver 2360 (as depicted in FIG. 5). By surrounding either the actuation member 2315 or the focus driver 2360, the actuation member 2315 can move to exert the mechanical force on the focus driver 2360 while the seal member 2370 maintains the hermetic seal of the optics chamber 2340. As depicted, in some embodiments, the seal member 2370 can be configured as a metal bellows that is configured to move between a collapsed configuration and an expanded configuration in response to the control input. As depicted in FIGS. 7 and 10, the seal member 2370 in the collapsed configuration is referenced by element number 2370a, while, as depicted in FIGS. 8 and 10, the seal member 2370 in the expanded configuration is referenced by element number 2370b.

As depicted in FIG. 7, the seal member 2370 has a collapsed configuration when the focus driver 2360 is at the first focus position FP₁. Similarly, as depicted in FIG. 8, the seal member 2370 has an expanded configuration when the focus driver 2360 is at the second focus position FP₂. As such, the seal member 2370 is configured to move between the collapsed configuration and the expanded configuration when the control input moves the focus driver 2360 between the first focus position FP₁ and the second focus position FP₂. As a metal bellows, the seal member 2370 has a distal end 2371 and a proximal end 2372 that can be fixedly coupled (e.g., via a weld, a solder joint, a compression fit and/or an adhesive) to different components of the imaging system 2330 that are movable relative to one another. With the distal end 2371 being fixedly coupled to a first component (e.g., the housing 2342) while the proximal end 2372 is fixedly coupled to a second component (e.g., the focus driver 2360 or the actuation member 2315), the seal member 2370 can maintain the hermetic seal of the optics chamber 2340 as the second component moves relative to the first component. The ability of the seal member 2370 to maintain the hermetic seal of the optics chamber 2340 as the components move relative to one another facilitates the control input affecting the focus driver 2360 without disrupting the hermetic seal of the optics chamber 2340. As such, the focusing element 2350 can be positioned as desired by the operator of the imaging instrument 2300 via a mechanical input while the elements of the imaging system 2330 within the optics chamber 2340 are isolated from the environment surrounding the optics chamber 2340.

Referring still to FIGS. 7 and 8, in some embodiments, the housing 2342 can include a flange portion 2344. The flange portion 2344 can be monolithically formed with the housing 2342 or separately formed and coupled to a remaining portion of the housing 2342, which can include additional segments (e.g., halves). The distal end 2371 of the seal member 2370 can be fixedly coupled to the flange portion 2344. The flange portion 2344, and the distal end 2371 of the seal member 2370 coupled thereto, is maintained at a fixed longitudinal position. Additionally, the focus driver 2360 can include a proximal portion 2361 that extends longitudinally through the flange portion 2344. The proximal end 2372 of the seal member 2370 can be fixedly coupled to the proximal portion 2361 of the focus driver 2360. Accordingly, as the focus driver 2360 moves longitudinally in response to the force applied by the actuation member 2315, the proximal end 2372 of the seal member 2370 moves longitudinally in concert with the proximal portion 2361. In other words, when the proximal portion 2361 of the focus driver 2360 moves a specified distance in the proximal direction in response to a tension force applied by the actuation member 2315, the proximal end 2372 of the seal member 2370 also moves the same distance in the proximal direction while the distal end 2371 remains at the fixed longitudinal position of the flange portion 2344. The movement of the proximal end 2372 while the distal end 2371 remains at the fixed longitudinal position transitions the seal member 2370 from the collapsed configuration to the expanded configuration. It should be appreciated that in some embodiments, the proximal end 2372 can be fixedly coupled to the actuation member 2315 in lieu of the proximal portion 2361.

As previously described, the boundary of the optics chamber 2340 defines a hermetically sealed volume V (represented in FIG. 7 by the dashed line). However, due to the expandable nature of the seal member 2370, the volume V of the optics chamber 2340 can change in accordance with the configuration of the seal member 2370. For example, in some embodiments, the optics chamber 2340 has a first volume when the seal member 2370 is in the collapsed configuration and a second volume when the seal member 2370 is in the expanded configuration. The second volume can be greater than the first volume. Said another way, the volume V of the optics chamber 2340 increases as the focus driver 2360 moves from the first focus position FP₁ with the seal member 2370 in a collapsed configuration to the second focus position FP₂ with the seal member in the expanded configuration. It should be appreciated that the other elements of the imaging system 2330 that form the boundary of the optics chamber 2340, such as the housing 2342, the distal tip 2314, the sapphire window 2384, the image sensor 2850, and the sealed couplings therebetween, remain in fixed positions relative to one another. As such, changes in the magnitude of the volume V of the optics chamber 2340 are dependent on the longitudinal positioning of the focus driver 2360 or an actuation member 2315 and the configuration of the seal member 2370. It should further be appreciated that in both the first volume and the second volume, and transitions therebetween, the optics chamber is hermetically sealed.

Referring again to FIG. 5, in some embodiments, the image sensor is operably coupled to a circuit board 2852. The circuit board 2852 can be positioned within the channel 2311 of the elongated body 2310. As shown, the circuit board 2852 is outside of the optics chamber 2340. In other embodiments, however, the circuit board 2852 can be positioned within the optics chamber 2340. The circuit board 2852 can be configured to process the electrical signals generated by the image sensor 2850 and transmit the processed signals to a display and/or a controller. The circuit board 2052 can also include additional sensors, such as a position sensor 2854. The position sensor 2054 can be used determine the longitudinal position of the focus driver 2360 and/or the actuation member 2315.

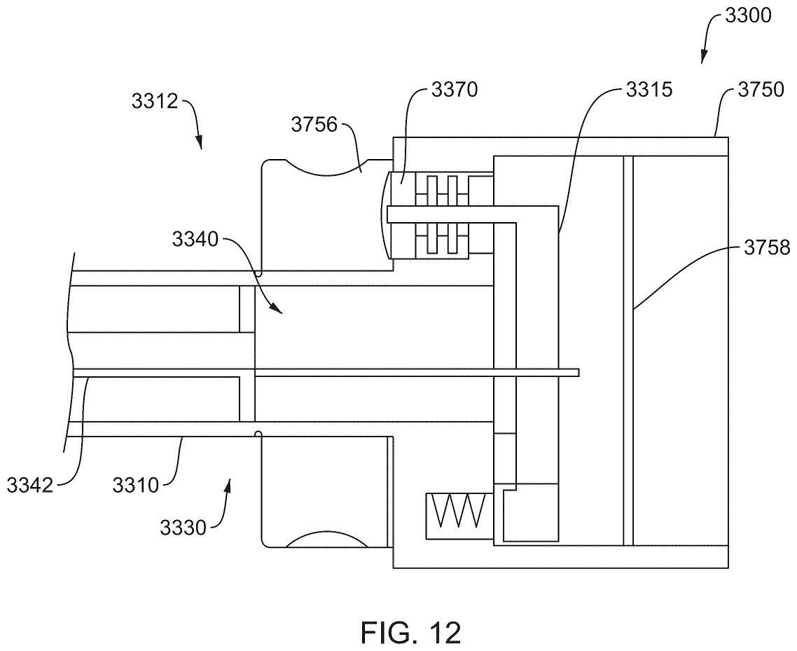

FIG. 12 is a diagrammatic view of a proximal portion of another imaging instrument 3300 that can be employed to image a target location. The imaging instrument 3300 can include any of the elements and components as described herein with reference to imaging instrument 1300 and/or imaging instrument 2300. In some embodiments, the imaging instrument 3300 or any of the components therein can be configured as a handheld endoscope for use in imaging a target location (e.g., a surgical site) in a space constrained environment. In some embodiments, the imaging instrument 3300 or any of the components therein are optionally parts of a surgical system that performs minimally invasive surgical procedures, and which can include a manipulator unit, a series of kinematic linkages, a series of cannulas, or the like.

In some embodiments, the imaging instrument 3300 includes an elongated body 3310 extending longitudinally between a proximal end portion 3312 and a distal end portion (not shown). As described above with reference to the imaging instrument 2300, the distal end portion is coupled to a distal tip. A proximal mechanical assembly 3750 is coupled to the proximal end portion 3312 of the elongated body 3310. The proximal mechanical assembly 3750 is configured to be coupled to a receiving unit (not shown). The proximal mechanical assembly 3750 is also configured to receive a control input from a user of the imaging instrument. For example, in some embodiments, the user of the imaging instrument 3300 can manipulate a first input wheel (not shown) to affect an orientation of the distal tip and/or a second input wheel 3756 to affect a focal distance of the imaging instrument 3300. In some embodiments, the imaging instrument 3300 is affected by the surgical system to image the target location.

As depicted, in some embodiments, the imaging instrument 3300 includes an imaging system 3330 that is positioned within the elongated body 3310 and partially within the proximal mechanical assembly 3750. Accordingly, the imaging system 3330 can extend along the longitudinal length of the elongated body 3310 to the distal portion of the elongated body 3310. In particular, the imaging system 3330 includes an optics chamber 3340 that extends the length of the elongated body 3310 and is at least partially within the proximal mechanical assembly 3750. The optics chamber 3340 (i.e., the boundary of the optics chamber 3340) is defined, at least in part, by a housing 3342 that extends the length of the elongated body 3310. In some embodiments, the boundary of the optics chamber 3340 is further defined by a portion of the proximal mechanical assembly 3750, a bulkhead 3758 positioned within the proximal mechanical assembly 3750, a seal member 3370, an actuation member 3315, and the sealed couplings therebetween. The sealed couplings between the various components defining the optics chamber 3340 can, for example, be glue joints, welds, and/or solders. In order to isolate the components of the imaging system 3330 positioned therein from moisture at the target location TL and/or encountered during post-procedure processing, the optics chamber 3340 is hermetically sealed. In other words, the volume defined by the optics chamber 3340 is fluidically isolated from the surrounding environment such that liquids, vapors, and/or gases are precluded from passing between the volume defined by the optics chamber 3340 and the surrounding environment. By maintaining the hermetic seal of the optics chamber 3340, the effect of moisture (e.g., fogging or condensation) on the optical components positioned therein is eliminated. By extending the housing 3342 the length of the elongated body 3310, various electronic components (not shown) can be positioned within the housing 3342 and isolated from the environment surrounding the optics chamber 3340.

As depicted in FIG. 12, the actuation member 3315 can be in contact with the second input wheel 3756 such that a rotation of the second input wheel 3756 causes a longitudinal movement of the actuation member 3315. As described previously with regards to the imaging instrument 2300, the movement of the actuation member 3315 can be used modify the focus distance of the imaging instrument 3300 via the application of a mechanical force. A hermetic seal between a portion of the proximal mechanical assembly 3750 and the actuation member 3315 can be established by the seal member 3370 positioned therebetween. The seal member 3370 can be configured to maintain the hermetic seal as the actuation member 3315 is moved longitudinally by the second input wheel 3756. Accordingly, the seal member 3370 can maintain the hermetic seal of the optics chamber 3341 of the control input affects the positioning of a focusing element (e.g., focusing element 1350 and/or focusing element 2350). To further facilitate maintenance of the hermetic seal as the actuation member 3315 is moved, a proximal boundary of the optics chamber 3340 can be formed by a bulkhead 3758 positioned within the proximal mechanical assembly 3750.

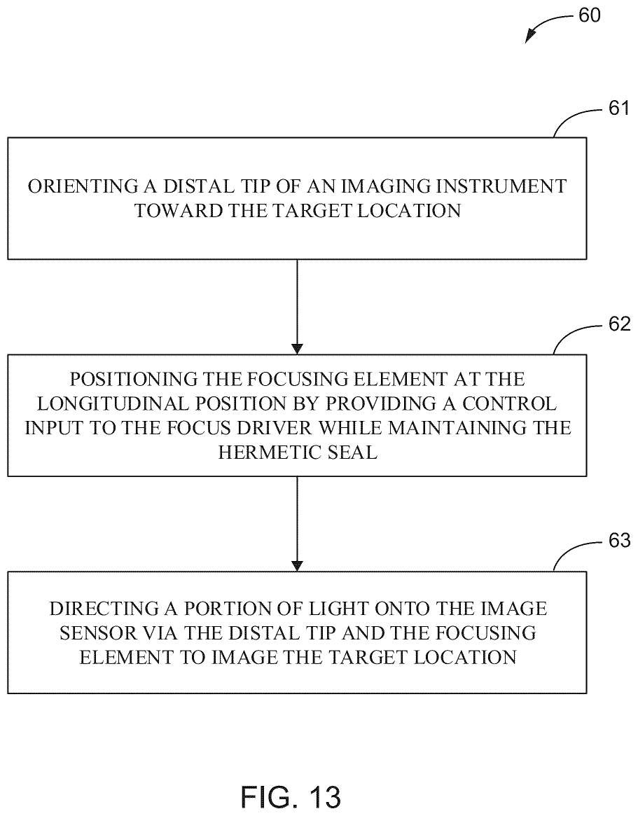

FIG. 13 is a flow chart of a method 60 of imaging a target location according to an embodiment. The method 60 may, in an embodiment, be performed via an imaging instrument, such as imaging instrument 2300 as described herein with reference to FIGS. 2-11. However, it should be appreciated that in various embodiments, aspects of the method 60 may be accomplished via additional embodiments of the imaging instrument 2300 or components thereof, such as imaging instrument 1300 or imaging instrument 3300 as described herein. Accordingly, the method 60 may be implemented on any suitable device as described herein. Thus, the method 60 is described below with reference to the imaging instrument 2300 as previously described, but it should be understood that the method 60 can be employed using any of the imaging instruments described herein.

As depicted at 61, the method 60 includes orienting a distal tip of an imaging instrument toward the target location. The imaging instrument includes a channel extending longitudinally through an elongated body between a proximal end portion of the elongated body and a distal end portion of the elongated body coupled to the distal tip and an imaging system positioned within the elongated body. The imaging system includes an optics chamber defined at least in part by a housing and which is hermetically sealed. A focusing element is movably positioned within the optics chamber, and an image sensor is operably coupled to the focusing element. Additionally, a focus driver is positioned within the optics chamber and operably coupled to the focusing element. The focus driver is configured to move a longitudinal position of the focusing element in response to a control input. Further, a seal member is coupled to the housing and configured to maintain the hermetic seal of the optics chamber. As depicted at 62, the method 60 includes positioning the focusing element at the longitudinal position by providing a control input to the focus driver while maintaining the hermetic seal. Additionally, as depicted at 63, the method 60 includes directing a portion of light onto the image sensor via the distal tip and the focusing element to image the target location.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the components of an instrument as described herein can be constructed from any material, such as medical grade stainless steel, nickel alloys, titanium alloys or the like. Further, any of the seal members, housings, or other components described herein can be constructed from multiple pieces that are later joined together. For example, in some embodiments, a seal member can be constructed by joining together separately constructed components. In other embodiments however, any of the seal members, housings, or components described herein can be monolithically constructed.

Although various embodiments have been described as having particular features and/or combinations of components, other embodiments are possible having a combination of any features and/or components from any of the embodiments as discussed above. Aspects have been described in the general context of medical devices, and more specifically surgical instruments, but inventive aspects are not necessarily limited to use in medical devices.

1. An imaging instrument, comprising:
an elongated body defining a channel extending longitudinally through the elongated body between a proximal end portion of the elongated body and a distal end portion of the elongated body, the distal end portion coupled to a distal tip; and
an imaging system positioned within the elongated body, the imaging system including:
an optics chamber defined at least in part by a housing, the optics chamber being hermetically sealed,
a focusing element movably positioned within the optics chamber,
an image sensor operably coupled to the focusing element,
a focus driver positioned within the optics chamber and operably coupled to the focusing element, the focus driver being configured to move a longitudinal position of the focusing element in response to a control input, and
a seal member coupled to the housing, the seal member configured to maintain a hermetic seal of the optics chamber while the control input affects the focus driver.

2. The imaging instrument of claim 1, further comprising:
a fiber-optic bundle positioned within the channel, the fiber-optic bundle including an input end operably coupled to a light source and an output end positioned at the distal tip, at least a portion of the fiber-optic bundle configured to be in fluid communication with an environment surrounding the imaging instrument.

3. The imaging instrument of claim 1, further comprising:
a proximal mechanical assembly coupled to the proximal end portion of the elongated body, the proximal mechanical assembly being configured to receive the control input; and
an actuation member positioned within the elongated body and coupled between the proximal mechanical assembly and the focus driver, the actuation member being configured to apply a mechanical force to the focus driver in a longitudinal direction in response to the control input.

4. The imaging instrument of claim 1, wherein:
the seal member is configured to move between a collapsed configuration and an expanded configuration when the control input moves the focus driver between a first focus position and a second focus position.

5. (canceled)

6. The imaging instrument of claim 4, wherein:
the optics chamber has a first volume when the seal member is in the collapsed configuration and a second volume when the seal member is in the expanded configuration; and
the second volume is greater than the first volume.

7. The imaging instrument of claim 1, wherein:
the housing includes a flange portion;
the focus driver includes a proximal portion that extends longitudinally through the flange portion; and
the seal member has a distal end that is coupled to the flange portion and a proximal end that is coupled to the proximal portion of the focus driver.

8. The imaging instrument of claim 1, wherein:
the seal member is a sealing assembly, the sealing assembly including at least one O-ring; and
the at least one O-ring surrounds either a proximal portion of the focus driver or an actuation member.

9. The imaging instrument of claim 1, further comprising:
an energy storage device positioned between a portion of the housing and a portion of the focus driver, the energy storage device being configured to exert a biasing force on the focus driver.

10. The imaging instrument of claim 1, further comprising:
a plurality of optical elements positioned to establish an optical axis from a target location, through the focusing element.

11. The imaging instrument of claim 10, wherein:
the plurality of optical elements includes a prism that is positioned between the focus driver and the focusing element.

12. The imaging instrument of claim 11, wherein:
the imaging system includes a movable lens holder;
the movable lens holder includes a middle portion extending between a distal end and a proximal end;
the focusing element is coupled to the distal end of the movable lens holder;
the proximal end of the movable lens holder is coupled to the focus driver; and
the middle portion of the movable lens holder is positioned radially outward of the prism such that the prism is between the movable lens holder and the image sensor.

13. The imaging instrument of claim 11, wherein:
the image sensor is displaced from an optical axis defined by the plurality of optical elements.

14. The imaging instrument of claim 10, wherein:
the plurality of optical elements includes a window that is positioned at the distal tip; and
the window defines a portion of the optics chamber.

15. The imaging instrument of claim 10, wherein:
the plurality of optical elements includes a lens group at a fixed longitudinal position that is distal of the focusing element and proximal of the distal tip.

16. (canceled)

17. A method of imaging a target location, the method comprising:
orienting a distal tip of an imaging instrument toward the target location, the imaging instrument including a channel extending longitudinally through an elongated body between a proximal end portion of the elongated body and a distal end portion of the elongated body coupled to the distal tip, and an imaging system positioned within the elongated body, the imaging system including:
an optics chamber defined at least in part by a housing, the optics chamber being hermetically sealed,
a focusing element movably positioned within the optics chamber,
an image sensor operably coupled to the focusing element,
a focus driver positioned within the optics chamber and operably coupled to the focusing element, the focus driver being configured to move a longitudinal position of the focusing element in response to a control input, and
a seal member coupled to the housing, the seal member configured to maintain the hermetic seal of the optics chamber;
positioning the focusing element at the longitudinal position by providing a control input to the focus driver while maintaining the hermetic seal; and
directing a portion of light onto the image sensor via the distal tip and the focusing element to image the target location.

18. (canceled)

19. The method of claim 17, wherein:
the imaging instrument includes a proximal mechanical assembly coupled to the proximal end portion of the elongated body and an actuation member positioned within the elongated body and coupled between the proximal mechanical assembly and the focus driver; and
the method includes:
providing a control input to the proximal mechanical assembly, and
maintaining the hermetic seal of the optics chamber while applying a mechanical force to the focus driver in a longitudinal direction via the actuation member in response to the control input.

20. The method of claim 19, wherein the longitudinal position of the focusing element is a first longitudinal position and the imaging system has a first focus distance when the focus driver is at a first focus position, and the mechanical force is a tension force, the method further comprising:
applying the tension force to the focus driver to move the focus driver longitudinally from the first focus position to a second focus position that is proximal to the first focus position, the focusing element being in a second longitudinal position and the imaging system having a second focus distance when the focus driver is at the second focus position, the second focus distance being greater than the first focus distance.

21. The method of claim 19, further comprising:
moving the focus driver between a first focus position and a second focus position via the mechanical force applied by the actuation member; and
maintaining the hermetic seal of the optics chamber while transitioning the seal member between a collapsed configuration when the focus driver is at the first focus position and an expanded configuration when the focus driver is at the second focus position.

22. (canceled)

23. The method of claim 17, wherein:
the imaging instrument includes an energy storage device positioned between a portion of the housing and a portion of the focus driver; and
the method includes exerting a biasing force on the focus driver in a longitudinal direction, the biasing force positioning the focusing element at a default longitudinal position.

24. (canceled)

25. (canceled)

26. The method of claim 17, wherein:
the imaging system includes a plurality of optical elements positioned to establish an optical axis from a target location, through the focusing element, and onto the image sensor;
the plurality of optical elements includes a prism that is positioned between the focus driver and the focusing element;
the imaging system includes a movable lens holder that has a middle portion extending between a distal end and a proximal end, the focusing element is coupled to the distal end of the movable lens holder and the proximal end is coupled to the focus driver, the middle portion of the movable lens holder is positioned radially outward of the prism such that the prism is between the movable lens holder and the image sensor; and
the method includes:
applying a force to the proximal end of the movable lens holder via the focus driver, the force is applied at a longitudinal position that is proximal relative to the prism, and
moving the middle portion of the movable lens holder longitudinally relative to the prism in response to the applied force to move the focusing element between a first longitudinal position and a second longitudinal position, the focusing element being positioned distally relative to the prism.
